(19) United States
(12) Patent Application Publication
(10) Pub. No.: US 2026/0008986 A1
(43) Pub. Date: Jan. 8, 2026

(54) Culture vessel system

(71) Applicant: Joseph G. Cremonese, Greensburg, PA (US)

(72) Inventor: Joseph G. Cremonese, Greensburg, PA (US)

(21) Appl. No.: 18/761,479

(22) Filed: Jul. 2, 2024

(51) Int. Cl. C12M 1/00; C12M 1/04

(52) U.S. Cl. CPC C12M 23/38; C12M 23/24; C12M 29/00; C12M 29/20

(57) ABSTRACT

A culture vessel system includes a growth vessel, a feed vessel, and a cap assembly. The growth vessel includes an open end, a first side wall that is cylindrical, and a first end wall that is dome-shaped. The feed vessel includes a second side wall, a second end wall, a third end wall, and a mouth portion projecting from the second end wall and including a passage to an interior of the feed vessel. The feed vessel is removably connected to the growth vessel with the open end adjacent the third end wall. The third end wall includes an input valve for passage of cell seed culture and liquid nutrient medium from the feed vessel to the growth vessel and an output valve for passage of gases from the growth vessel to the feed vessel. The cap assembly includes a gas passage and is removably connected to the mouth portion.

### CROSS-REFERENCE TO RELATED APPLICATIONS Not Applicable

### STATEMENT REGARDING FEDERALLY SPONSORED RESEARCH OR DEVELOPMENT Not Applicable

### THE NAMES OF THE PARTIES TO A JOINT RESEARCH AGREEMENT Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to cell culturing systems and more particularly pertains to a new culture vessel system for improved growth and scaling up of cell species for production of biological products, such as hyaluronic acid and monoclonal antibodies. “Fed-batch” culturing is a process that starts with small volume cultures that are fed additional nutrients with metered addition to manage proliferating cell cultures. In most cases, an Erlenmeyer flask with a large flat bottom and inwardly sloping side walls will be used in order to maximize surface area of the liquid nutrient medium therein to improve transfer of oxygen into the liquid nutrient medium. Since the Erlenmeyer flask narrows toward the top, scaling up of batches by adding more liquid nutrient medium is not possible because the surface area decreases. In addition, the Erlenmeyer flask requires a large amount of initial material. The new culture vessel system uses a growth vessel that is cylindrical with a dome-shaped bottom, which allows for the use of a small volume of liquid for a starting culture and allows for scaling up by addition of more liquid nutrient medium in a controlled manner without decreasing surface area, which results in more efficient growth of stable cells and less waste of cells. The culture vessel system also allows for feeding of gases useful for the cell growth process, such as oxygen for aerobic processes and nitrogen or carbon dioxide for anaerobic processes, as well as degassing of unwanted gases, such as carbon dioxide formed during aerobic processes. The cylindrical vessel also allows for well behaved swirling of the liquid, which is not possible with the inwardly sloping walls of the Erlenmeyer flask.

### (2) DESCRIPTION OF RELATED ART INCLUDING INFORMATION DISCLOSED UNDER 37 CFR 1.97 AND 1.98

The prior art relates to cell culturing systems. The prior art, as best understood, does not disclose a culture vessel system that includes a growth vessel having a dome-shaped bottom and a feed vessel that has an input valve for feed of seed culture and an output valve for passage of gases from said growth vessel to said feed vessel.

### BRIEF SUMMARY OF THE INVENTION

An embodiment of the disclosure meets the needs presented above in a culture vessel system generally comprising a growth vessel, a feed vessel, and a cap assembly. The growth vessel includes an open end, a first side wall that is cylindrical, and a first end wall that is dome-shaped. The feed vessel includes a second side wall, a second end wall, a third end wall, and a mouth portion projecting from the second end wall and including a passage to an interior of the feed vessel, wherein the feed vessel is removably connected to the growth vessel with the open end adjacent the third end wall, and wherein the third end wall includes an input valve to permit passage of cell seed culture and liquid nutrient medium from the feed vessel to the growth vessel and an output valve to permit passage of gases from the growth vessel to the feed vessel. The cap assembly includes a gas passage therein and is removably connected to the mouth portion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded view of a culture vessel system according to an embodiment of the disclosure.

FIG. 2 is a side view of an embodiment of the disclosure.

FIG. 3 is a perspective view of an embodiment of the disclosure.

FIG. 4 is a perspective view of an embodiment of the disclosure.

FIG. 5 is an exploded perspective view of an embodiment of the disclosure.

FIG. 6 is an exploded perspective view of an embodiment of the disclosure.

FIG. 7 is an exploded cross-sectional view of an embodiment of the disclosure.

FIG. 8 is a cross-sectional view of an embodiment of the disclosure.

FIG. 9 is an exploded perspective view of an embodiment of the disclosure in use.

FIG. 10 is an exploded side view of an embodiment of the disclosure.

FIG. 11 is a perspective view of an embodiment of the disclosure in use.

FIG. 12 is a side view of an embodiment of the disclosure in use.

FIG. 13 is a side view of an embodiment of the disclosure in use.

### DETAILED DESCRIPTION OF THE INVENTION

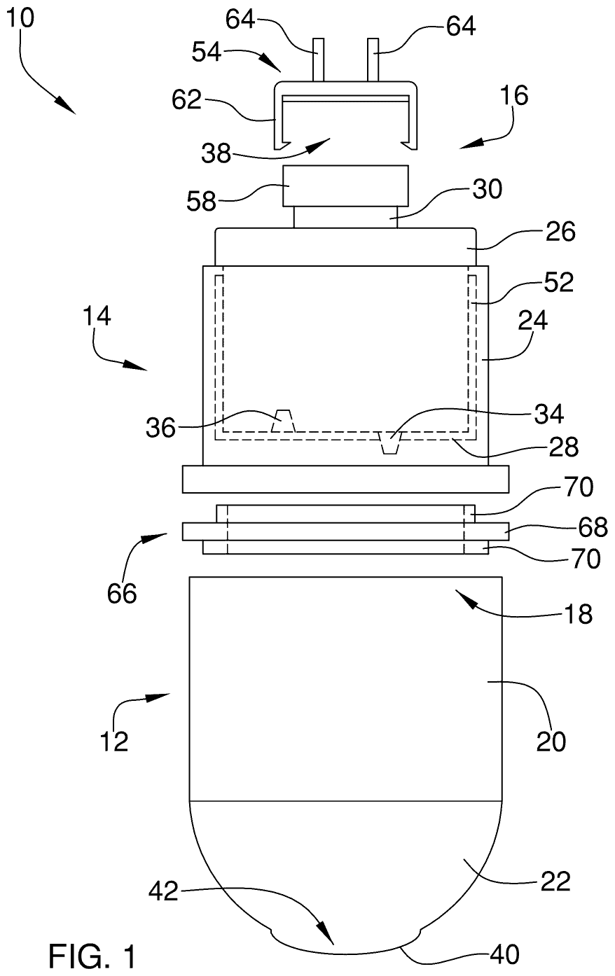

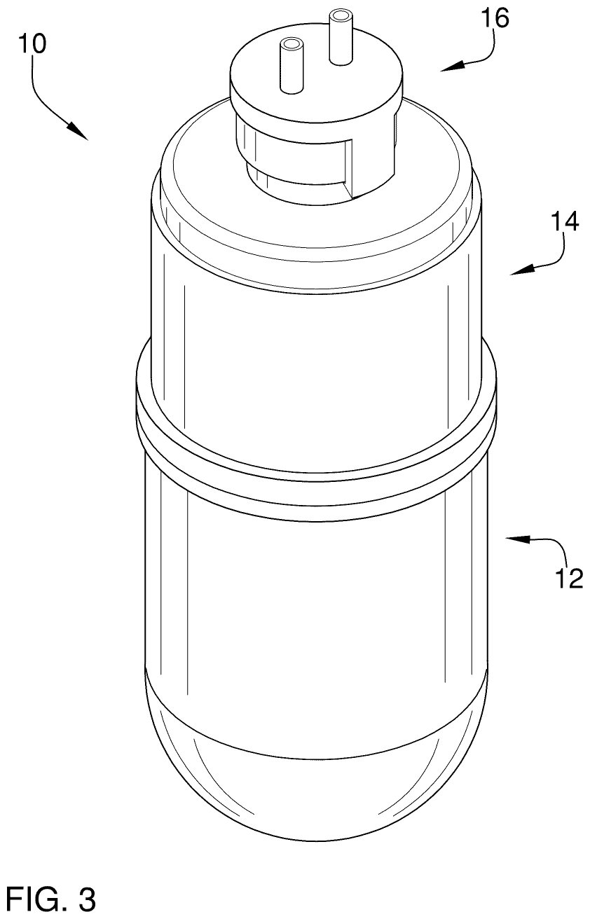

With reference now to the drawings, and in particular to FIGS. 1 through 13 thereof, a new culture vessel system embodying the principles and concepts of an embodiment of the disclosure and generally designated by the reference numeral 10 will be described.

As best illustrated in FIGS. 1 through 13, the culture vessel system 10 generally comprises a growth vessel 12, a feed vessel 14, and a cap assembly 16. The growth vessel 12 includes an open end 18, a first side wall 20 that is cylindrical, and a first end wall 22 that is dome-shaped. The feed vessel 14 includes a second side wall 24, a second end wall 26, a third end wall 28, and a mouth portion 30 projecting from the second end wall 26 and including a passage 32 to an interior of the feed vessel 14, wherein the feed vessel 14 is removably connected to the growth vessel 12 with the open end 18 adjacent the third end wall 28, and wherein the third end wall 28 includes an input valve 34 to permit passage of cell seed culture and liquid nutrient medium from the feed vessel 14 to the growth vessel 12 and an output valve 36 to permit passage of gases from the growth vessel 12 to the feed vessel 14. The cap assembly 16 includes a gas passage 38 therein and is removably connected to the mouth portion 30.

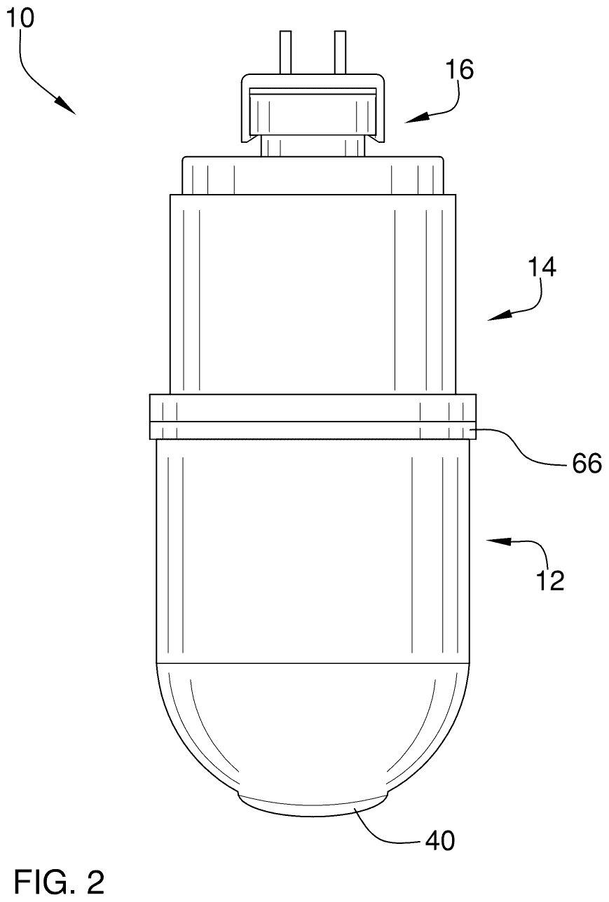

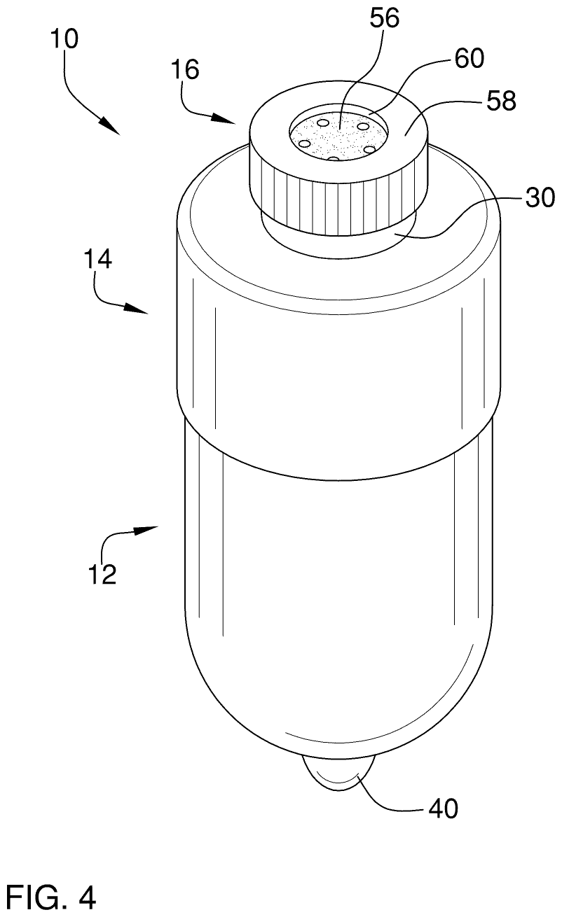

The first end wall 22 includes a convex projection 40 positioned at a distal central section of the first end wall 22, which convex projection 40 forms an indentation 42 inside the growth vessel 12 designed to accumulate cell seed culture and cells therein. This accumulation of cell seed culture allows for the use of a rather limited volume of cell seed culture and liquid nutrient medium. In an Erlenmeyer flask, for example, a large amount of liquid nutrient medium has to be inserted into the Erlenmeyer flask to cover the large flat bottom of the Erlenmeyer flask. In general, cells tend to consume as much nutrients as are available, which can result in very fast growth. As a result, the cells can proliferate too quickly, thereby resulting in death of some or most of these cells. By using a smaller starting amount as allowed by the indentation 42, such as, for example, around 30 milliliters, the nutrient levels can be better controlled for a steady growth of healthy usable cells and ultimately a scaling up to a very large volume of cells not possible with an Erlenmeyer flask or other known containers. As shown in FIGS. 1 and 2, the convex projection 40 has a height substantially less than a width thereof. As shown in FIGS. 4 through 6, alternatively the convex projection 40 has a height greater than a width thereof.

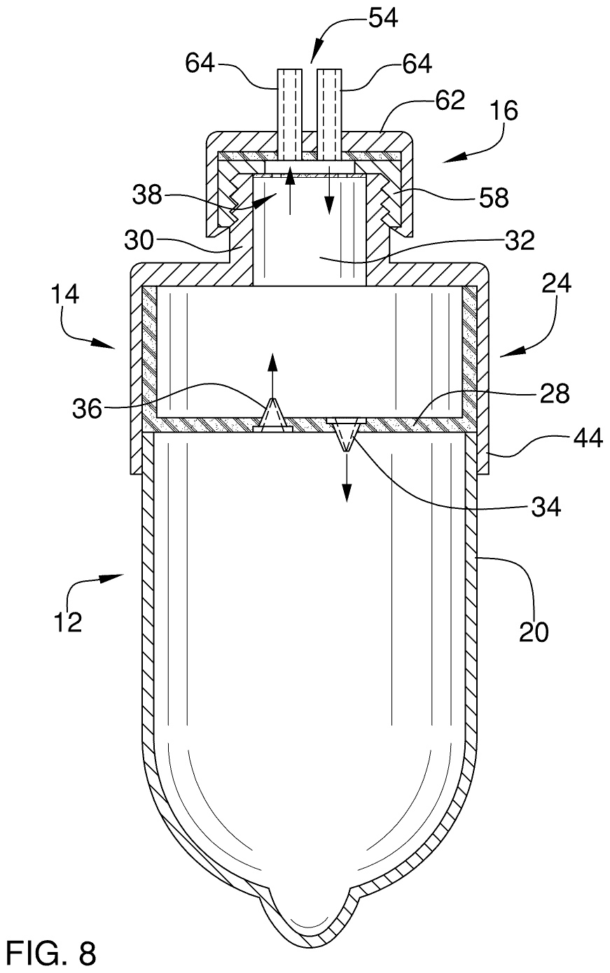

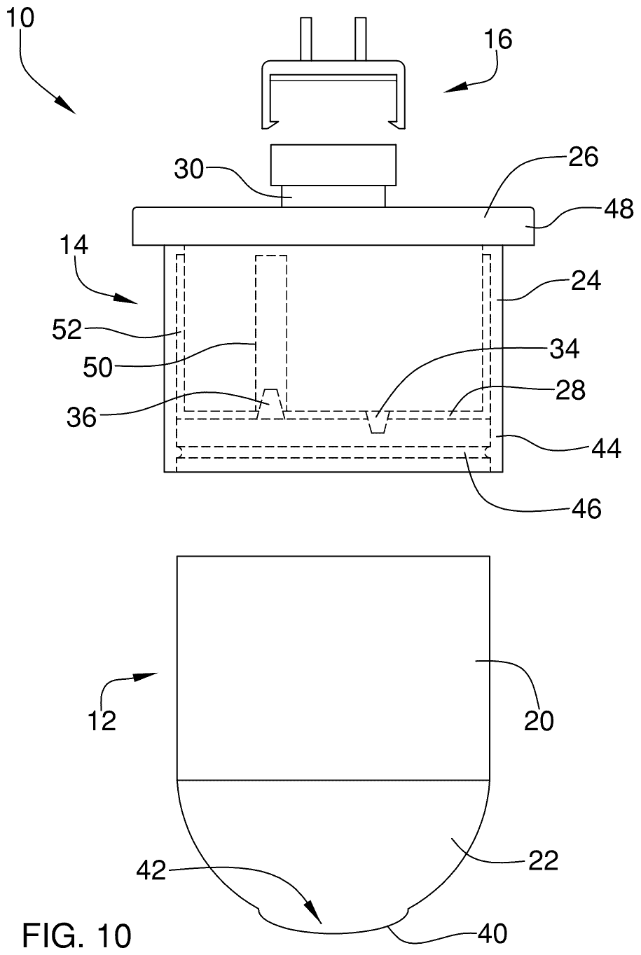

As best seen in FIG. 8, in one possible embodiment the second side wall 24 is cylindrical and has a projecting portion 44 that extends beyond the third end wall 28 and has an inner diameter that is greater than an outer diameter of the first side wall 20 at the open end 18 such that the growth vessel 12 is partially inserted into the feed vessel 14 when connected. The inner diameter of the projecting portion 44 can be essentially equivalent to the outer diameter of the first side wall 20 to removably connect the feed vessel 14 and the growth vessel 12 by friction fit. As shown in FIG. 10, in one possible embodiment the culture vessel system 10 further includes a resilient sealing ring 46, such as a silicone or rubber ring, positioned on an interior surface of the projecting portion 44 and designed to engage the first side wall 20 to seal and removably connect the feed vessel 14 and the growth vessel 12 by friction fit.

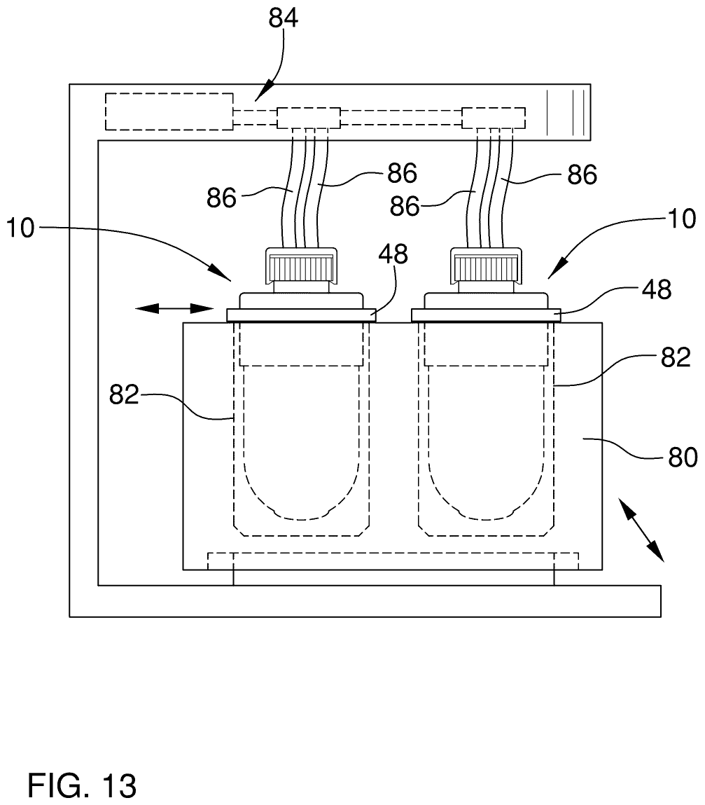

Also as shown in FIG. 10, in one possible embodiment, the second end wall 26 is disk-shaped and includes a shoulder portion 48 that projects annularly beyond the second side wall 24. The shoulder portion 48 is designed to support the feed vessel 14, and the growth vessel 12 connected thereto, in a suspended manner in a receptacle 82 of a culture vessel handling machine 80, such as is shown, for example, in FIG. 13.

Also as shown in FIG. 10, in one possible embodiment, the feed vessel 14 includes a tube 50 connected to and extending from the output valve 36 and terminating adjacent the second end wall 26. The tube 50 allows for evacuation of gases from the growth vessel 12 even if there is liquid nutrient medium in the feed vessel 14.

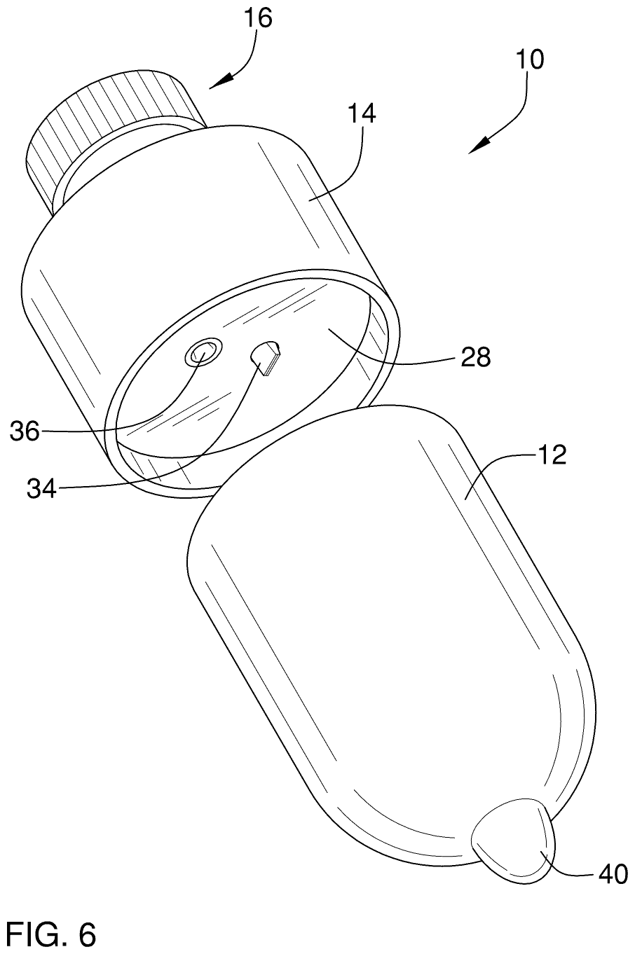

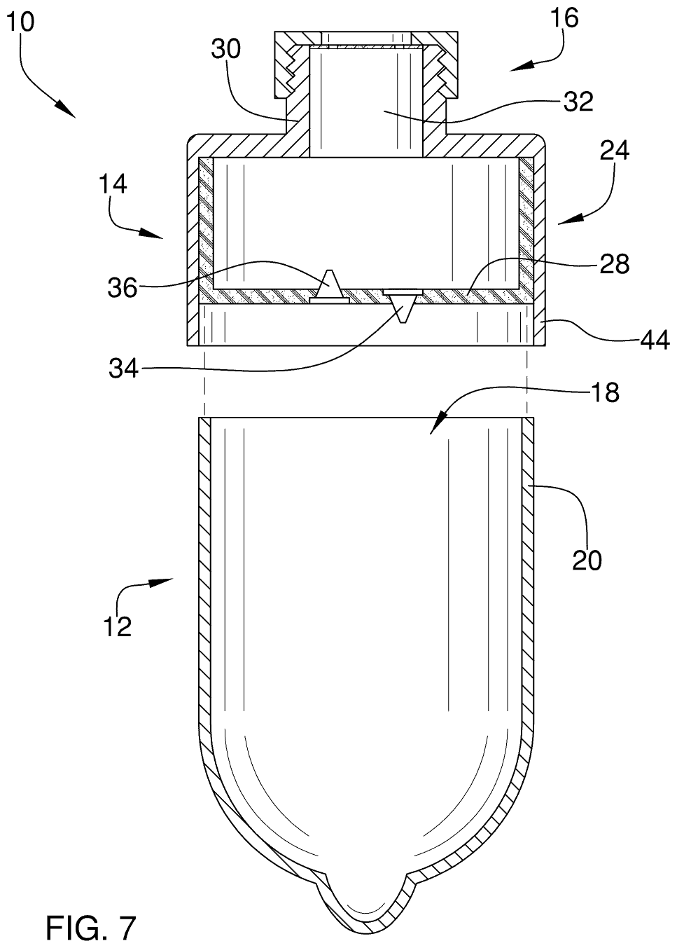

The input valve 34 and the output valve 36 are designed as one-way valves. In one possible embodiment, best shown in FIGS. 6 and 7, the feed vessel 14 includes a silicone liner 52 that is cylindrical, wherein the third end wall 28 is also silicone and is integrally formed with the silicone liner 52 to form a cup-like structure removably mounted within the second side wall 24. Each of the input valve 34 and the output valve 36 is a duckbill valve made of silicone and integrally formed in the third end wall 28. While a duckbill valve is shown in the figures, other valve structures are within the scope of the disclosure. In one possible embodiment, the silicone liner 52 is omitted and the third end wall 28 is integrally formed with the second side wall 24. In another possible embodiment, silicone liner 52 is permanently adhered or connected to the second side wall 24.

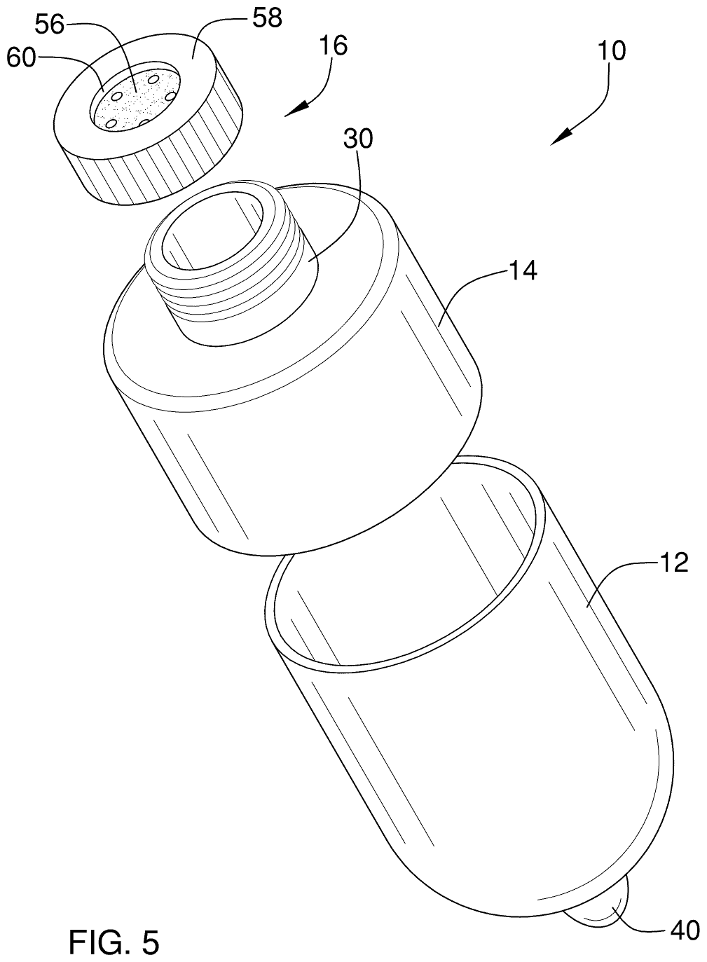

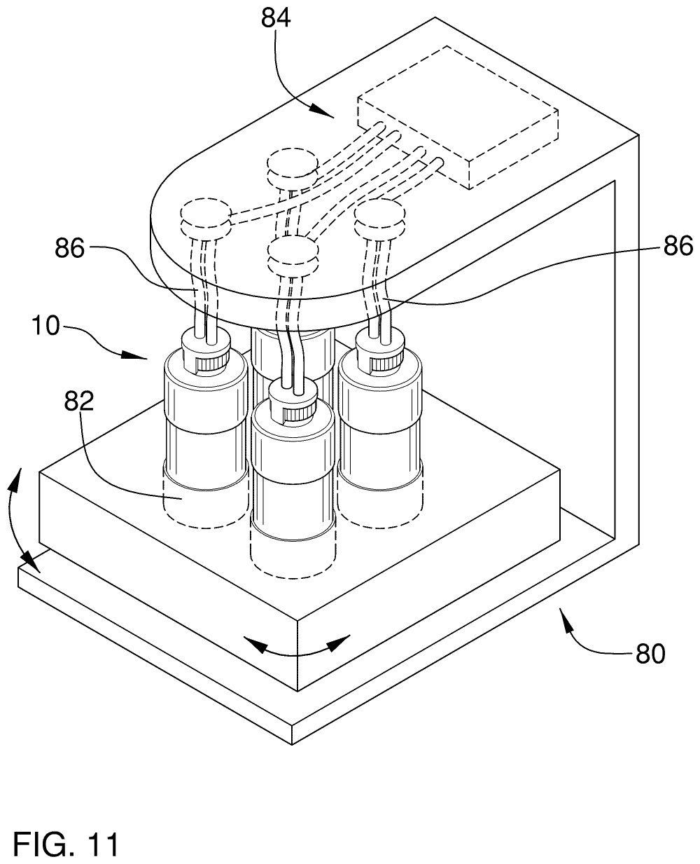

As shown in FIGS. 1 and 8, the gas passage 38 of the cap assembly 16 includes a hose connector 54 designed to operatively connect the cap assembly 16 to hoses 86 of an air pump assembly 84, as shown for example in FIG. 11, to permit creation of positive and negative pressure inside the growth vessel 12 and the feed vessel 14. In the exemplary embodiment shown in the figures, the gas passage 38 further includes a gas-permeable membrane 56, as best shown in FIG. 4. With reference again to FIG. 1, the cap assembly 16 can include a first cap portion 58 that is removably connected over the mouth portion 30, such as by a screw-threaded connection as shown in FIG. 5 or other suitable connection, such as a friction fit or snap fit. The gas-permeable membrane 56 is positioned in an orifice 60 in the first cap portion 58. The cap assembly 16 also includes a second cap portion 62 removably connected over the first cap portion 58, such as by a snap fit as shown in FIG. 2 or other suitable connection, such as a screw thread or friction fit. The hose connector 54 is positioned on an outer surface of the second cap portion 62. In the exemplary embodiment shown, the hose connector 54 is in the form of two projecting tubes 64 that can be connected to or inserted into the hoses 86 of the air pump assembly 84, though in an alternative embodiment the projecting tubes 64 could be integrally formed with the hoses 86, such that the second cap portion 62 is permanently connected to the air pump assembly 84. In the embodiment shown in FIG. 11, the air pump assembly 84 is part of a culture vessel handling machine 80, and the hoses 86 are flexible to permit oscillating movement of the individual culture vessel systems 10. There are two hoses 86 for each individual culture vessel system 10, one for conducting gases in and one for vacuuming gases out. It should be understood that the exemplary embodiment of the air pump assembly 84 is shown relatively schematically and could be executed in any number of ways as is known in gas flow technology. In addition, the air pump assembly 84 could be mounted on or integrally formed as part of the culture vessel handling machine 80, or could be a stand-alone unit adjacent the culture vessel handling machine 80.

In one possible embodiment shown in FIG. 1, the culture vessel system 10 further includes a connecting ring 66 positioned between and to removably connect the feed vessel 14 and the growth vessel 12 by friction fit. The connecting ring 66 includes an annular flange 68 positioned between the ends of the growth vessel 12 and the feed vessel 14 and two insert portions 70, one each inserted into the growth vessel 12 and the feed vessel 14. The connecting ring 66 could be made of a resilient material, such as silicone or rubber.

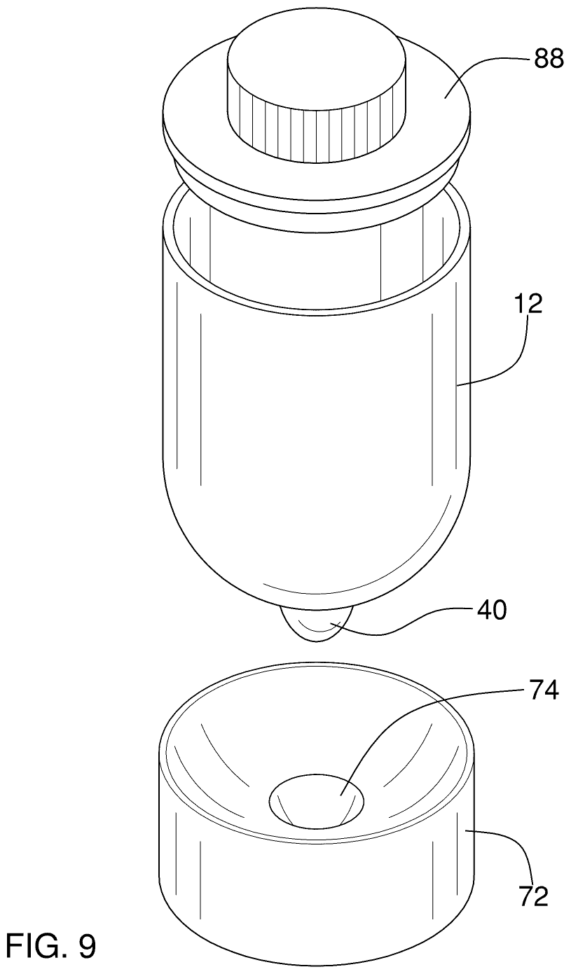

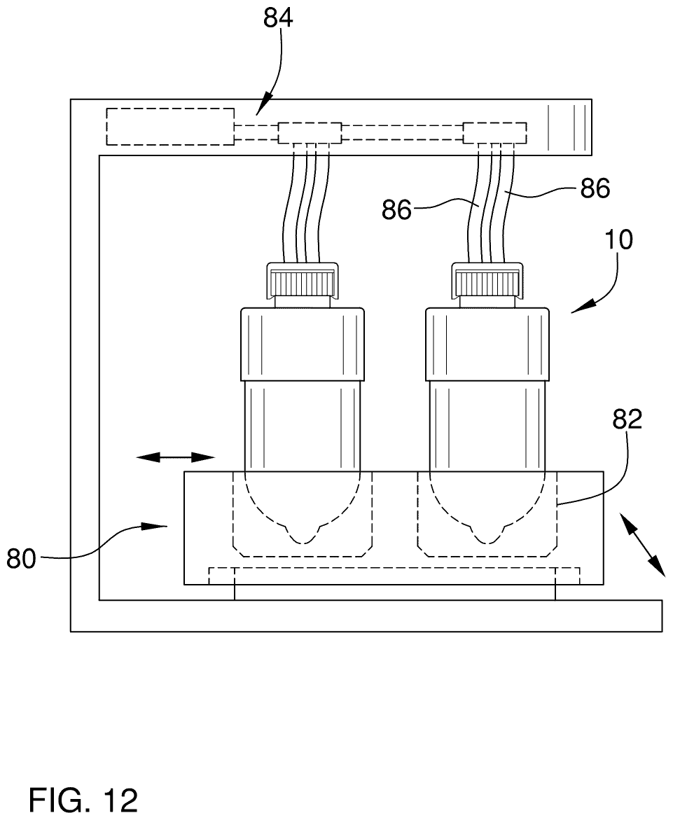

In one possible embodiment shown in FIG. 9, the culture vessel system 10 further includes a cylindrical cushion 72 designed to be inserted into a receptacle of a centrifuge to support and protect the first end wall 22. The cylindrical cushion 72 includes an opening 74 therein designed to receive the convex projection 40. After a desired volume of cells has been grown, the feed vessel 14 is removed and a centrifuge cover 88 is placed over the open end 18 of the growth vessel 12. The growth vessel 12 is then placed in a centrifuge to separate the cells for harvesting. Standard centrifuges are not designed to accommodate a vessel like the growth vessel 12 with the projection 40, so the cylindrical cushion 72 could be inserted into the centrifuge to protect the projection 40 from being damaged. The cushion 72 could also be utilized in the culture vessel handling machine 80, such as in the embodiment shown in FIG. 12 where the receptacles 82 are shorter than those shown in FIG. 13.

In one possible embodiment, the feed vessel 14 is a one-piece plastic structure and the growth vessel 12 is a one-piece plastic structure. The plastic could be polycarbonate, polyethylene, or polypropylene. The plastic could be a disposable material to permit a user to throw away the feed vessel 14 and the growth vessel 12 after one use, rather than having to clean and sterilize them.

It should be noted that any of the components in any of the embodiments disclosed herein could be used or integrated into a different embodiment in any reasonable combination thereof. Identical or equivalent parts marked in one figure may not be marked in all figures for simplicity.

To use the culture vessel system 10, the user connects the growth vessel 12 and the feed vessel 14 and thereby closes the growth vessel 12. The user then inserts through the mouth portion 30 of the feed vessel 14 an amount of cell seed culture and liquid nutrient medium into the feed vessel 14. The user places the cap assembly 16 onto the mouth portion 30 and closes the feed vessel 14. The user can then operatively connect the cap assembly 16 to the air pump assembly 84, such as in the exemplary embodiment in FIG. 11. The user can activate the air pump assembly 84 to create a positive pressure in the feed vessel 14 and thereby force the cell seed culture and the liquid nutrient medium through the input valve 34 and into the growth vessel 12. The cell seed culture accumulates by gravity at the bottom of the first end wall 22, such as in the indentation 42. The user can place the growth vessel 12 in a receptacle 82, or multiple growth vessels 12 in multiple receptacles 82, of the culture vessel handling machine 80 designed to move the growth vessel 12 in a reciprocating and translating movement. The user then activates the culture vessel handling machine 80 and selectively moves the growth vessel 12, which causes a swirling movement of the liquid nutrient medium and causes the liquid nutrient medium to spread up and across interior surfaces of the first side wall 20. This swirling results in a maximization of the surface area of the liquid to improve gas transfer into and out of the liquid. The user can also create a negative pressure in the growth vessel 12 and the feed vessel 14 with the air pump assembly 84 and thereby withdraw gases from the growth vessel 12 through the output valve 36 and the feed vessel 14 through the mouth portion 30. For example, if the user is performing an aerobic growth process and the user wishes to evacuate carbon dioxide from the growth vessel 12, the user can activate the air pump assembly 84 to draw out such carbon dioxide. The user can selectively repeat one or more of the preceding steps in a fed-batch process to grow a desired amount of cells. For example, after a desired amount of cells have been grown in a first batch, the user can remove the cap assembly 16 and insert more cell seed culture and/or liquid nutrient medium into the feed vessel 14, then repeat the subsequent steps to grow more cells in the growth vessel 12. The user can vary the process as desired depending on the cells to be grown, such as by varying the amounts of cell seed culture and liquid nutrient medium, varying the types of gases, varying the pressures, and so forth. In one possible embodiment, the user can start with a combined volume of the cell seed culture and the liquid nutrient medium of approximately 30 milliliters and increase the combined volume in batches to approximately 400 milliliters in the growth vessel 12. This type of small starting amount and large scale up is not possible with current containers and flasks. In one possible embodiment, the growth vessel 12 can be designed to hold from 100 milliliters to one liter of liquid, including tens and hundreds of milliliters in that range.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A culture vessel system comprising:
a growth vessel comprising an open end, a first side wall that is cylindrical, and a first end wall that is dome-shaped;
a feed vessel comprising a second side wall, a second end wall, a third end wall, and a mouth portion projecting from said second end wall and comprising a passage to an interior of said feed vessel, wherein said feed vessel is removably connected to said growth vessel with said open end adjacent said third end wall, and wherein said third end wall comprises an input valve to permit passage of cell seed culture and liquid nutrient medium from said feed vessel to said growth vessel and an output valve to permit passage of gases from said growth vessel to said feed vessel; and
a cap assembly comprising a gas passage therein and being removably connected to said mouth portion.

2. The culture vessel system of claim 1, wherein said first end wall comprises a convex projection disposed at a distal central section of said first end wall, which convex projection forms an indentation inside said growth vessel configured to accumulate cell seed culture and cells therein.

3. The culture vessel system of claim 2, wherein said convex projection has a height substantially less than a width thereof.

4. The culture vessel system of claim 2, wherein said convex projection has a height greater than a width thereof.

5. The culture vessel system of claim 1, wherein said second side wall is cylindrical and has a projecting portion that extends beyond said third end wall and has an inner diameter that is greater than an outer diameter of said first side wall at said open end such that said growth vessel is partially inserted into said feed vessel when connected.

6. The culture vessel system of claim 5, wherein said inner diameter of said projecting portion is essentially equivalent to said outer diameter of said first side wall to removably connect said feed vessel and said growth vessel by friction fit.

7. The culture vessel system of claim 5, wherein the culture vessel system further comprises a resilient sealing ring disposed on an interior surface of said projecting portion and configured to engage said first side wall to seal and removably connect said feed vessel and said growth vessel by friction fit.

8. The culture vessel system of claim 1, wherein said second end wall is disk-shaped and comprises a shoulder portion that projects annularly beyond said second side wall, wherein said shoulder portion is configured to support said feed vessel, and said growth vessel connected thereto, in a suspended manner in a receptacle of a culture vessel handling machine.

9. The culture vessel system of claim 1, wherein said feed vessel comprises a tube connected to and extending from said output valve and terminating adjacent said second end wall.

10. The culture vessel system of claim 1, wherein each of said input valve and said output valve is a one-way valve.

11. The culture vessel system of claim 1, wherein said feed vessel comprises a silicone liner that is cylindrical, wherein said third end wall comprises silicone and is integrally formed with said silicone liner to form a cup-like structure removably mounted within said second side wall, and wherein each of said input valve and said output valve comprises a duckbill valve made of silicone and integrally formed in said third end wall.

12. The culture vessel system of claim 1, wherein said gas passage comprises a hose connector configured to operatively connect said cap to hoses of an air pump assembly to permit creation of positive and negative pressure inside said growth vessel and said feed vessel.

13. The culture vessel system of claim 12, wherein said gas passage further comprises a gas-permeable membrane.

14. The culture vessel system of claim 13, wherein said cap assembly comprises a first cap portion removably connected over said mouth portion, wherein said gas-permeable membrane is disposed in an orifice in said first cap portion, and a second cap portion removably connected over said first cap portion, wherein said hose connector is disposed on an outer surface of said second cap portion.

15. The culture vessel system of claim 1, wherein:
the culture vessel system further comprises a connecting ring disposed between and to removably connect said feed vessel and said growth vessel by friction fit;
said connecting ring comprises an annular flange positioned between the ends of the growth vessel and the feed vessel and two insert portions, one each inserted into the growth vessel and the feed vessel; and
said connecting ring comprises a resilient material.

16. The culture vessel system of claim 1, wherein:
the culture vessel system further comprises a cylindrical cushion configured to be inserted into a receptacle of a centrifuge to support and protect said first end wall; and
said cylindrical cushion comprises an opening therein configured to receive said convex projection.

17. The culture vessel system of claim 1, wherein said feed vessel is a one-piece plastic structure and said growth vessel is a one-piece plastic structure.

18. A method of using the culture vessel system of claim 1, comprising the steps of:
connecting said growth vessel and said feed vessel and closing said growth vessel;
inserting through said mouth portion of said feed vessel an amount of cell seed culture and liquid nutrient medium into said feed vessel;
placing said cap assembly onto said mouth portion and closing said feed vessel;
operatively connecting said cap assembly to an air pump assembly;
creating a positive pressure in said feed vessel with the air pump assembly and forcing the cell seed culture and the liquid nutrient medium through said input valve and into said growth vessel, wherein said cell seed culture accumulates by gravity at the bottom of said first end wall;
placing said growth vessel in a receptacle of a culture vessel handling machine configured to move said growth vessel in a reciprocating and translating movement;
selectively moving said growth vessel and causing a swirling movement of the liquid nutrient medium and causing the liquid nutrient medium to spread up and across interior surfaces of said first side wall;
creating a negative pressure in said growth vessel and said feed vessel with the air pump assembly and withdrawing gases from said growth vessel through said output valve and said feed vessel through said mouth portion; and
selectively repeating one or more of the preceding steps in a fed-batch process to grow a desired amount of cells.

19. The method of claim 18, wherein said method comprises starting with a combined volume of the cell seed culture and the liquid nutrient medium of approximately 30 milliliters and increasing the combined volume in batches to approximately 400 milliliters in said growth vessel.

20. A culture vessel system comprising:
a growth vessel comprising an open end, a first side wall that is cylindrical, and a first end wall that is dome-shaped, wherein:
said first end wall comprises a convex projection disposed at a distal central section of said first end wall, which convex projection forms an indentation inside said growth vessel configured to accumulate cell seed culture and cells therein, wherein one of:
said convex projection has a height substantially less than a width thereof,
said convex projection has a height greater than a width thereof, said growth vessel is a one-piece plastic structure;
a feed vessel comprising a second side wall, a second end wall, a third end wall, and a mouth portion projecting from said second end wall and comprising a passage to an interior of said feed vessel, wherein said feed vessel is removably connected to said growth vessel with said open end adjacent said third end wall, and wherein said third end wall comprises an input valve to permit passage of cell seed culture and liquid nutrient medium from said feed vessel to said growth vessel and an output valve to permit passage of gases from said growth vessel to said feed vessel, wherein:
said second side wall is cylindrical and has a projecting portion that extends beyond said third end wall and has an inner diameter that is greater than an outer diameter of said first side wall at said open end such that said growth vessel is partially inserted into said feed vessel when connected,
said inner diameter of said projecting portion is essentially equivalent to said outer diameter of said first side wall to removably connect said feed vessel and said growth vessel by friction fit,
the culture vessel system further comprises a resilient sealing ring disposed on an interior surface of said projecting portion and configured to engage said first side wall to seal and removably connect said feed vessel and said growth vessel by friction fit,
said second end wall is disk-shaped and comprises a shoulder portion that projects annularly beyond said second side wall, wherein said shoulder portion is configured to support said feed vessel, and said growth vessel connected thereto, in a suspended manner in a receptacle of a culture vessel handling machine,
said feed vessel is a one-piece plastic structure,
said feed vessel comprises a tube connected to and extending from said output valve and terminating adjacent said second end wall,
each of said input valve and said output valve is a one-way valve,
said feed vessel comprises a silicone liner that is cylindrical, wherein said third end wall comprises silicone and is integrally formed with said silicone liner to form a cup-like structure removably mounted within said second side wall, and wherein each of said input valve and said output valve comprises a duckbill valve made of silicone and integrally formed in said third end wall; and
a cap assembly comprising a gas passage therein and being removably connected to said mouth portion, wherein:
said gas passage comprises a gas-permeable membrane,
said gas passage comprises a hose connector configured to operatively connect said cap to hoses of an air pump assembly to permit creation of positive and negative pressure inside said growth vessel and said feed vessel,
said cap assembly comprises a first cap portion removably connected over said mouth portion, wherein said gas-permeable membrane is disposed in an orifice in said first cap portion, and a second cap portion removably connected over said first cap portion, wherein said hose connector is disposed on an outer surface of said second cap portion.
